(19) United States
(12) Patent Application Publication
(10) Pub. No.: US 2007/0203533 A1
(43) Pub. Date: Aug. 30, 2007

(54) Implantable medical device for restoration of neurological function impaired by peripheral neuropathy

(75) Inventors: Andy Ofer Goren, Newport Beach, CA (US); Yehuda G. Goren, Scotts Valley, CA (US); Peter Novak, Jamaica Plain, MA (US); Elliott J. Stein, Morristown, NJ (US); Christopher Chi-Chuen Chen, Wallace, CA (US); Amy Morningstar, Scotts Valley, CA (US); David A. Eckhous, Long Beach, CA (US)

Correspondence Address: THELEN REID BROWN RAYSMAN & STEINER LLP, P. O. BOX 640640, SAN JOSE, CA, 95164-0640, US

(73) Assignee: BioQ, Inc., Newport Beach, CA (US)

(21) Appl. No.: 11/650,571

(22) Filed: Jan. 5, 2007

Related U.S. Application Data
(63) Continuation-in-part of application No. 11/512,739, filed on Aug. 29, 2006.
(60) Provisional application No. 60/712,976, filed on Aug. 30, 2005.
(60) Provisional application No. 60/831,035, filed on Jul. 13, 2006.

(51) Int. Cl. A61N 1/00; A61H 1/00; A61B 5/103

(52) U.S. Cl. 607/49; 600/595; 601/46; 607/144

(57) ABSTRACT

An implantable device for treating a patient using sensory substitution includes a wearable article in which are disposed one or more sensors for detecting the phase of the gait cycle of the patient, a controller for receiving signals from the sensors indicative of the phase of the gait, and one more stimulators for stimulating the patient based on signals from the controller that are issued in response to the sensor signals.

### CROSS-REFERENCE TO RELATED APPLICATIONS

This application is a continuation-in-part of U.S. patent application Ser. No. 11/512,739, filed on Aug. 29, 2006, entitled “Medical Device for Restoration of Neurological Function Impaired by Peripheral Neuropathy,” which claims the benefit of U.S. provisional patent application No. 60/712,976, filed on Aug. 30, 2005, entitled “Medical Device for Treatment of Balance and Gait Disorders Using Sensory Substitution,” and of U.S. provisional patent application No. 60/831,035, filed on Jul. 13, 2006, entitled “Therapeutic Device for Prevention of Ulcerations Using Sensory Substitution,” each of which are incorporated herein by reference.

### BACKGROUND OF THE INVENTION

1. Field of the Invention

The invention relates to the treatment of peripheral neuropathy disorders.

2. Description of Related Art

A major problem facing patients suffering from peripheral neuropathy as well as the general aging population is the increased risk of falls and development of ulcerations during walking. During human gait, transmission of cutaneous feedback from the feet is essential for maintaining normal gait and balance. Non-nociceptive cutaneous feedback from the feet is normally transduced via mechanoreceptors at the sole and transmitted via the afferent nerve fibers to the central nervous system.

It is well documented in the medical literature that peripheral neuropathy results in functional loss of nerve fibers which is usually irreversible and has no medical treatment currently available. The loss of nerve fibers is characterized by severe sensory deficit of vibrational and tactile perception and subsequent degradation of gait and balance leading to an increased incidence of falls and fractures.

Another problem facing patients suffering from peripheral neuropathy is the increased risk of developing foot ulcerations. The decrease in cutaneous feedback from the feet of patients suffering from peripheral neuropathy and the associated gait impairment results in the development of abnormal planar pressure during human gait. Abnormal planar pressure results in abnormal repetitive stress to the feet and thus increases the risk of developing foot ulcerations.

Various devices have been proposed to attempt to improve abnormal cutaneous feedback from the feet in patients with neuropathy. One approach stimulates the patient's feet with “noise”—that is, random sub-threshold mechanical or electrical stimulation in order to reduce the threshold of cutaneous mechanoreceptors. A shortcoming of this approach is that the stimulation intensity needs to adjusted individually for each patient and the long term effectiveness of the treatment remains unclear. In another approach the patient's feet are stimulated using supra-threshold vibratory mechanical stimulation in order to overcome the increased stimulus threshold of the cutaneous mechanoreceptors. Shortcomings of this approach include the potential for nerve damage due to repetitive supra-threshold vibratory mechanical stimulation, the lack of effectiveness of the device in subjects with peripheral neuropathy, and the practical means of energizing a device embedded in a subject's shoe.

There therefore exist a need for a system that overcomes the limitations of previous approaches by providing a implantable, low cost, self contained device that stimulates a subject's tendon, ligament, bone or other tissue containing mechanoreceptors less affected by peripheral neuropathy in accordance with the phase of the gait cycle in order to treat balance and gait disorders and prevent falls and fractures as well as problems associated with abnormal planar pressure resulting in abnormal repetitive stress to the feet and increasing the risk of developing foot ulcerations

### SUMMARY OF THE INVENTION

The current invention makes use of the phenomenon of sensory substitution. Sensory substitution is a known neurological phenomenon whereby a subject with a failed or degraded mode of perception learns that an input signal from different sensory receptors (less affected by neuropathy or damage) in the subject's body are used to complement the failed or degraded perception. In accordance with one embodiment of the invention, there is provided a device for providing neural sensory substitution. The device includes one or more sensors configured to generate acceleration signals in response to a human gait during the human gait cycle, a controller configured to determine phases of the human gait cycle using the acceleration signals and to issue control signals in accordance with the determined gait phases, and one or more stimulators configured to stimulate the patient using the device in response to the control signals.

In accordance with another embodiment of the invention, there is provided a device for treating a gait disorder of a patient. The device includes an article or component that is wearable by (or implantable in body of) the patient, one or more sensors coupled to the article and configured to generate acceleration signals in response to the gait of the patient, a controller configured to determine phases of the gait of the patient using the acceleration signals and to issue control signals in accordance with the determined phases, and one or more stimulators configured to stimulate the patient in response to the control signals.

In accordance with yet another embodiment of the invention, there is provided a device for reducing the risk and/or preventing the formation of foot ulcerations in diabetic patients. The device includes an article component that is wearable by (or implantable in the body of) the patient, one or more sensors coupled to the article and configured to generate acceleration signals in response to the gait of the patient, a controller configured to determine phases of the gait of the patient using the acceleration signals and to issue control signals in accordance with the determined phases, and one or more stimulators configured to stimulate the patient in response to the control signals.

Also disclosed herein is a method for treating a gait disorder of a patient and reducing the likelihood of sustaining falls and fractures. The method includes generating a first set of electric signals indicative of gait-induced motion, generating a representation of a gait cycle based on the first set of electric signals, and using said gait cycle representation to provide feedback to the patient.

### BRIEF DESCRIPTION OF THE SEVERAL VIEWS OF THE DRAWINGS

Many advantages of the present invention will be apparent to those skilled in the art with a reading of this specification in conjunction with the attached drawings, wherein like reference numerals are applied to like elements, and wherein:

FIG. 1 is perspective view of a device 10 worn on the leg of a patient and utilizing sensory substitution;

FIG. 2 is a cross-sectional view of the device 10 of FIG. 1;

FIG. 3 is a schematic view of components of a system comprising device 10 of FIG. 1;

FIG. 4 is schematic view of a device in the form of a footwear 25 utilizing sensory substitution

FIG. 5 is a schematic diagram of an implantable device; and

FIG. 6 is a flow diagram showing a method for treating a gait disorder of a patient.

### DETAILED DESCRIPTION OF THE INVENTION

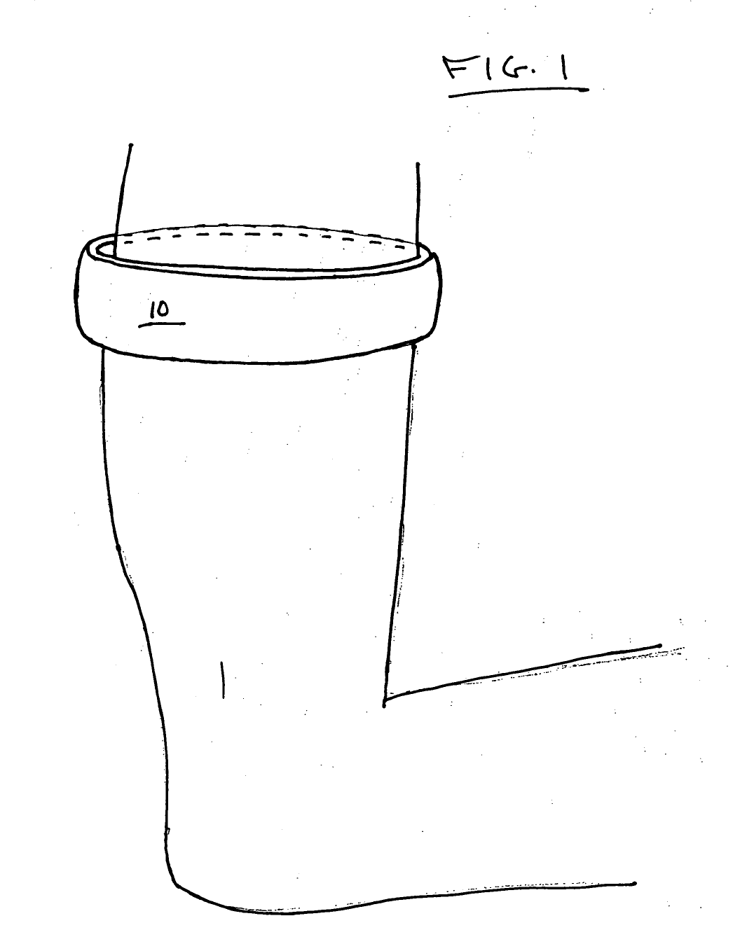

FIG. 1 is a perspective view of a therapeutic device in the form of a cuff 10 worn on the leg of a patient for treating balance or gait disorders as well as reduction of risk of ulcerations, falls, and fractures. The cuff or similar worn article may be in the form of a conforming, comfortable elastic band of suitable durability and compatibility with the skin of the wearer. While the preferred location for wearing the cuff is the leg, other places are also contemplated, such as the arm or wrist (bracelet), neck, sole of the foot, ankle, and so forth.

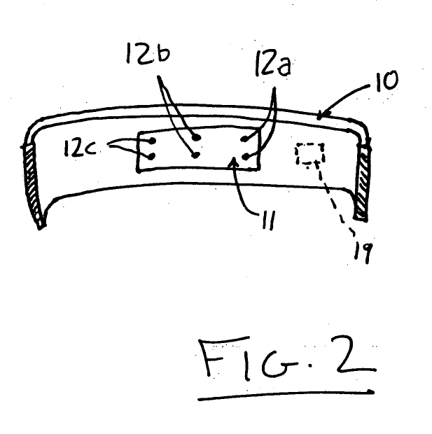

FIG. 2 is a cross-sectional cut-away view of cuff 10, showing a contact pad 11 on an interior surface of the cuff intended to make contact with the skin of the patient when the cuff is worn. Contact pad 11 has a set of six stimulators grouped in pairs 12a, 12b and 12c that are disposed respectively in anterior, central and posterior portions of the contact pad. It will be appreciated that the number, grouping and location of the stimulators are not critical. More or less than six may be used, and these may or may not be grouped in pairs, and may or may not be disposed symmetrically in the contact pad. The particular arrangement of stimulators should be selected such that optimum stimulation effect of the patient is achieved thereby. One example of a selectable arrangement of stimulators is a geometrical pattern that mimics the location of the contact points of the human foot relative to the ground during the human gait cycle. The stimulators 12a, 12b and 12c can be for example vibratory stimulators that provide mechanical supra-threshold neuronal stimulation to skin mechanoreceptors. Such stimulation can for example be vibration. Stimulators 12a, 12b and 12c can also be of a type that provides transcutaneous electrical stimulation to the skin mechanoreceptors. They can also provide electrical stimulation to at least one efferent nerve, in which case they can be implantable in the body of the patient proximal to the particular efferent nerve. They can also provide mechanical pressure to a body part of the patient, or provide auditory/hearing aid, visual, vibratory mechanical, olfactory, taste, heat/cold, or pain stimulation. More generally, the stimulators are configured to provide stimulation to any portion of the sensory system of the patient such that sensory substitution can occur. The sensory system should be understood to include receptors, neurons or afferent nerve fibers, which provide input upon which sensory substitution can be based.

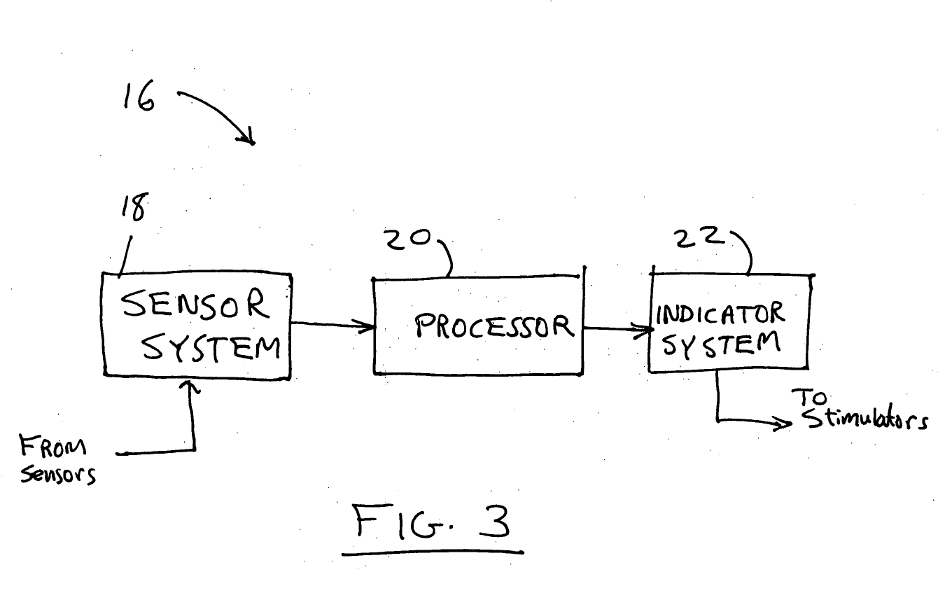

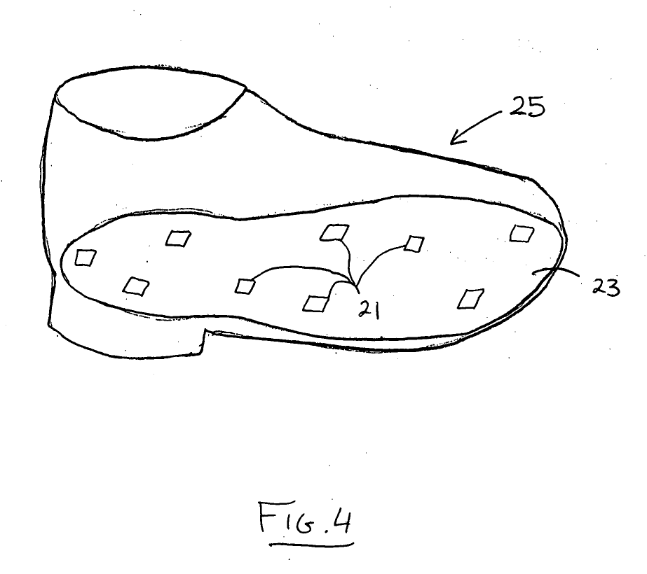

FIG. 3 is a schematic diagram of a therapeutic system 16 included with cuff 10. A sensor system 18 provides input signals to a controller or processor 20, which in turn activates an indicator system 22 accordingly. The processor 20 may be “hard-wired” to perform as desired, or it may be programmable such that its functions can be tailored to the particular patient's needs and the device fitted accordingly. In the preferred embodiment, the indicator system 22 includes the stimulators 12a, 12b and 12c. The sensor system 18 is designed to provide information to the processor 20 to thereby enable the processor to distinguish and/or predict various phases of the gait cycle. The gait cycle is the time between any two identical walking events during human walking. Each gait cycle is divided into a stance and swing period. The stance period constitutes 62 percent of the gait cycle and is composed of 5 phases: initial contact, loading response, midstance, terminal stance, and preswing. The swing period constitutes 38 percent of the gait cycle and is composed of 3 phases: initial swing, midswing, and terminal swing. Sensor system 18 includes one or more acceleration-measuring sensors (that is, accelerometers) 19 housed in cuff 10 (FIG. 2). Alternatively, sensors 19 may be housed in a separate device or cuff (not shown) worn by or implanted in the patient and communicating with the cuff 20 wirelessly or with a wire. The sensors 19 of sensor system 18 are designed to pick up accelerations (negative and/or positive) during the human gate cycle, caused for instance by the impact of parts of the foot, such as the heel or toes, against the ground, and/or accelerations of the foot during its swing between ground contacts, and/or accelerations induced by lifting of the foot from the ground. The information from the sensors 19, including the direction and magnitude of the accelerations and their point of occurrence for instance as coinciding with ground impact, is forwarded to the processor 20, which translates the information into an indication of the phase of the gait cycle. Alternatively, sensor system 18 can be in the form of one or more pressure-sensors 21 embedded in a specially-fitted portion 23 of a shoe 25 worn by the patient, as shown in FIG. 4. While portion 23 is shown to correspond to the insole of the shoe 25, other footwear components or portions of the shoe, in lieu of or in addition to the insole, can be so outfitted. In addition, the system 16 itself can be housed in a shoe or similarly-wearable device, dispensing with the need to provide cuff 10. Another possibility is in the form of a sock for example. The information from the sensors 21 is forwarded to the processor 20, which translates the information into a representation of the patient's gait cycle. Communication between the sensors 21 and processor 20 would preferably take place wirelessly, and suitable power sources, transmitters, and receivers (not shown) for effecting this, disposed in the shoe 23 and the cuff 10, would be provided as necessary. It may also be advantageous, depending on the application, to use sensors in the form of gyroscopes, or piezoelectric devices or the like.

The information from sensor system 18 as translated by processor 20 into the indication or representation of the patient's gait cycle, is used to effect selective activation of the indicators 22, and in particular, stimulators 12a, 12b and 12c, to thereby provide the patient with feedback regarding his/her position and possible magnitude in the gate cycle. The stimulators 12a, 12b and 12c are mapped to correspond to different regions of the foot, preferably but not necessarily in a correspondence with the portion of the foot that would normally be most activated during the particular phase of the gait cycle. Specifically, anterior stimulators 12a correspond to the front of the foot or the toes, and can be activated when this portion of the foot is for example determined by the processor 20 to be in contact with the ground, particularly during the push-off phase of the gait cycle. Central stimulators 12b can be activated when the foot is flat against the ground, for example during mid-stance. Posterior stimulators 12c can be activated during heel strike or initial contact. Of course, combinations of stimulators 12a-12c can be activated at various times during the gait cycle. Further, the activation can be suitably timed to account for impulse travel times, reaction times, and so forth in order to provide optimum effect. Further, as stated above, while three sets of stimulators are described, more or fewer sets, grouped differently and consisting of more or fewer than three can also be used. In addition, indicators other than or in addition to the stimulators can be used, including auditory and/or visual and/or vibratory indicators. Also as mentioned above, a suitable power supply would be provided in the cuff to drive system 16, and can include a rechargeable battery pack (not shown). Power can also be obtained from a non-battery source, or from an electromechanical source which converts kinetic energy into electrical energy.

The system 16 is designed to provide feedback to the patient to help the patient maintain balance or otherwise improve his/her gait and reduce the risk of falls and fractures. It is also intended to provide feedback to the patient in order to address the problem of foot ulcerations due to abnormal planar pressure. In addition, since the system uses sensory substitution by providing feedback to a different location from that from which information about the gait is normally derived physiologically, patients with a markedly reduced feeling, for example in their feet, can still benefit since they would receive information, through stimulators 12a, 12b and 12c, at the location of the cuff, which can be tailored to the patient's needs and is not limited to the leg location shown in FIG. 1.

Depending on the type of acceleration sensors 19 used, their location within cuff 10 may or may not be critical, based on the direction of motion of the patient's leg. Further, while described in terms of correcting gait disorders, it will be appreciated that balance or stance disorders can also be addressed. Sensors/acceleration detectors that can pick up patient motion in a lateral direction would be useful in such systems, particularly in a direction that is perpendicular or transverse to the gait direction, for example in the direction of “swaying” due to loss of balance.

An example of an accelerometer that can be used to detect balance and gait disorders in humans is a low-g accelerometer such as the ADXL203™ by Analog Devices. The ADXL203™ can detect acceleration components in up to 2 independent perpendicular axes. Each acceleration component can detect an acceleration in the range of +/−1.7 g. The ADXL203™ has a very high sensitivity of 1000 mV/g which is useful in the sway detection as well as a very low energy consumption of up to 2.1 mW power at 3 V battery source. Finally, the ADXL203™ is extremely light and compact size—that is, as small as 5 mm×5 mm×2 mm, and weighing less that 0.5 gram.

The stimulators 12a, 12b and 12c are selected to provide mechanical supra-threshold neuronal stimulation to the skin mechanoreceptors of the patient. Alternatively or in addition, the stimulators 12a, 12b and 12c can be selected to provide transcutaneous electrical stimulation to the skin mechanoreceptors. To optimize the effect of the stimulators, an adjustment mechanism may be provided to adjust the intensity of the stimulations they provide. Adjustment may also be desired so as to provide the patient with phase or magnitude information relating to the cycle. Further, intensity adjustment may be effected automatically by the controller or processor 20. The controller may be configured to activate and deactivate one or more of the stimulators in a temporal pattern to provide the wearer with phase information relating to the gait cycle. The phase information can also be indicated by using a pattern of stimulation frequencies.

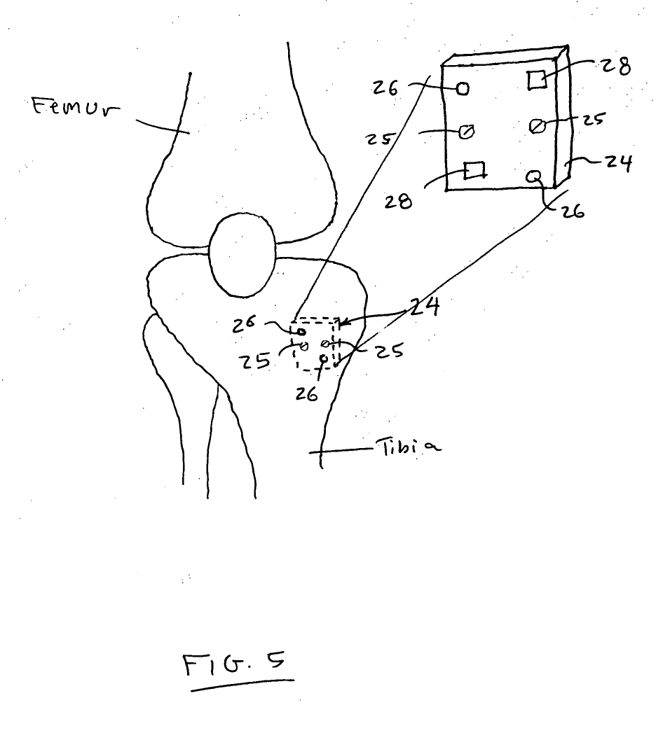

FIG. 5 schematically illustrates a therapeutic device 24 that is designed to be implantable in the body of the patient to provide the above-described sensory augmentation or substitution therapy. Device 24 can be implanted behind the tibia and attached to the bone, and is provided with a suitable attachment mechanism—which in the illustrated example includes through-holes (not shown) through which screws 25 are passed for threading into the bone. Other attachment mechanisms are possible, including staples, suturing or placement in any body cavity. Vibrational stimulators 26 can be used in device 24 to provide stimulation commensurate with the therapeutical regime contemplated in accordance with the above discussion. Specifically, the stimulators are configured to provide stimulation to any portion of the sensory system of the patient such that sensory substitution can occur. The sensory system should be understood to include receptors, neurons or afferent nerve fibers, which provide input that allows sensory substitution. Vibrational stimulators are suitable for taking advantage of the bone conduction phenomenon to augment their input, thereby requiring less power. That means that battery replacement is less frequent, which is important since implantation limits access to the device 24.

Device 24 can be implanted subcutaneously or can be implanted in any body cavity, bone, or interosseus membrane. The device 24 can be implanted through percutaneous or surgical incision procedure. The implant can be located in proximity to a bone or tendon and may include a bone attachment for optimal positioning. As mentioned above, the device 24 can be stapled or screwed to the bone for secure and optimal positioning. The device 24 can be implanted in the leg in proximity to the tibia bone; however, the device could also be implanted anywhere in the human body such as the hip, upper leg, bone, and so forth. The device 24 can be configured for vibratory stimulation to the bone and/or tendon and/or ligament and/or muscle mechanoreceptors and/or soft tissue so as to augment the missing sensory information. The bone is known to conduct vibrations and can be detected by the various mechanoreceptors. The device 24 can stimulate nerves, muscles, tendons, ligaments, soft tissue, periosteum or any combination thereof using electrical stimulation, mechanical stimulation, or acoustic stimulation. For example, the device 24 uses electrical stimulation to directly stimulate afferent nerves. A nerve in the calf may be used for this application.

Device 24 can comprise the entire therapeutic apparatus, including sensors 28 and processor or controller (not shown), or it can comprise merely a portion of it—for example the stimulator portion. In the latter case, the other portions containing the sensors and/or processor can be disposed elsewhere on or in the patient—for example in the a cuff, anklet, and so forth. The two components can then communicate wirelessly or through a hard-wired connection.

As described above, the processor operates to detect the phase of the gait cycle and/or the limb position in space and determine the optimal stimulation including frequency, duration, and amplitude of the stimulus. The sensors 28 can be acoustic sensors configured to detect acoustic signals indicative of limb motion and/or the phase of the gait cycle. For example, the heel strike phase produces a certain acoustic signal detectable by the sensors 28, and signals indicative thereof are conveyed to the processor to provide the necessary information. The sensors can also or alternatively be accelerometers, tilt meters, goniometers, gyroscopes, and so forth.

An additional advantage of the bone-attached implantation is that it can be used induce bone healing and/or bone mineral density increase. This healing application can be performed in a dedicated healing mode, or in combination with the above-described sensory augmentation therapy. The stimulation that can be applied for this purpose can be vibratory, mechanical, electrical, or acoustic, and use the same stimulators as that in the sensory augmentation therapy, or it can use different stimulators dedicated to the healing therapy.

Device 24 also includes a power source (not shown) for powering the various electronic components such as the sensors, stimulators and processor. The power source may be any conventional source, such as any of a variety of batteries that may or may not be rechargeable. In the latter case, recharging can be effected using conductors that may lead from the device 24 to the exterior of the patient's body and that are suitably configured to prevent infection or injury. Alternatively, recharging can be carried out using an inductive coupling between the battery and/or device 24 on one side, and a charger (not shown) on the other. In addition to communicating power in these manners described, it may be advantageous to program or reprogram the processor in device 24 in similar manner—namely, using suitably-configured conductors or inductively.

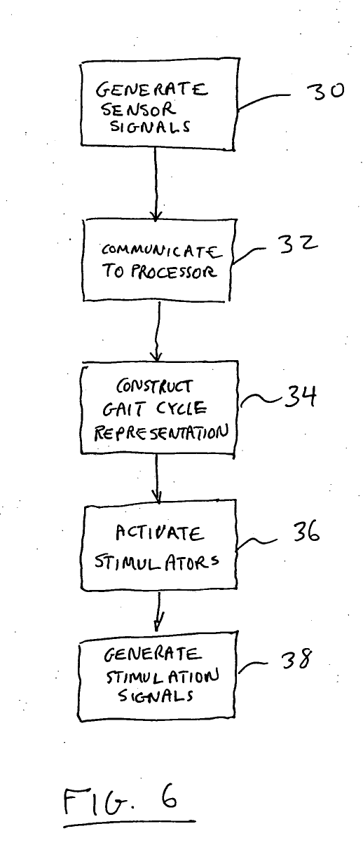

Consistent with the above description, a flow diagram of a method for correcting a gait disorder in a patient and reducing the likelihood of falls and fractures is shown in FIG. 6. At Step 30, a set of electrical signals are generated, for example using a sensor system such as system 18. The sensor system is configured such that the electrical signals are functions of the gait of the patient, with the sensors being suitably selected and placed so as to sense various gait-induced characteristics such as accelerations or decelerations, weight shifts, impacts, limb position or orientation changes, and so forth. The electrical signals are communicated to a processor or controller at Step 32. The processor then uses these signals to construct a representation of the gait cycle of the patient, at Step 34. The processor then activates, at Step 36, a set of one or more stimulators, such as stimulators 12a, 12b and 12c, which may be implanted in the body of the patient or disposed in a wearable housing which is optionally shared by the processor and/or the sensors of sensor system 18. The stimulators, upon activation, generate, at Step 38, stimulation signals or feedback designed to be sensed by the patient. Based on these stimulation signals, the patient can learn to adjust his/her gait, primarily by relying on the sensory substitution phenomenon.

The above are exemplary modes of carrying out the invention and are not intended to be limiting. It will be apparent to those of ordinary skill in the art that modifications thereto can be made without departure from the spirit and scope of the invention as set forth in the following claims.

1. An implantable device for providing neural sensory substitution to a patient, comprising:
one or more sensors configured to generate acceleration signals in response to a phase and/or a phase change of a human gait cycle;
a controller configured to determine phases of said human gait cycle using said acceleration signals and to issue control signals in accordance with said determined phases;
one or more stimulators configured to stimulate the sensory system of the patient in response to said control signals; and
an attachment mechanism configured to attach the implantable device to the patient.

2. The device of claim 1, wherein at least one stimulator is a vibrational stimulator.

3. The device of claim 2, wherein the stimulator is operative in a healing mode selected to induce bone healing and/or bone mineral density increase in the patient.

4. The device of claim 1, wherein the controller is programmable.

5. The device of claim 1, wherein the acceleration signals are generated based on lifting of a foot of the patient from ground during the human gait cycle.

6. The device of claim 1, wherein the acceleration signals are generated based on impact of a foot of the patient with ground during the human gait cycle.

7. The device of claim 1, wherein the acceleration signals are generated in response to acceleration in a direction that is transverse to a direction of the patient's gait.

8. The device of claim 1, wherein the controller selectively activates the stimulators based on prediction of phases of the gait of the patient.

9. The device of claim 1, further comprising:
a first component in which is disposed at least one sensor; and
a second component in which is disposed at least one stimulator,
wherein the controller is disposed in one of the first or second components and communicates wirelessly or via wired means with at least one sensor and/or at least one stimulator.

10. The device of claim 1, wherein at least one of the one or more sensors is a gyroscope.

11. The device of claim 1, wherein at least one of the one or more sensors is an accelerometer.

12. The device of claim 1, wherein at least one of the one or more sensors is a pieozoelectric sensor.

13. The device of claim 1, further comprising a rechargeable power source.

14. The device of claim 1, further comprising an electromechancial power source.

15. The device of claim 1, wherein the one or more stimulators are arranged in a geometrical pattern mimicking contact points of a human foot with ground during a human gait cycle.

16. The device of claim 1, said device configured for use to treat gait and balance disorders in peripheral neuropathy patients.

17. The device of claim 1, said device configured for use to prevent and/or reduce falls and/or fractures in neuropathy patients.

18. The device of claim 1, said device configured for use to reduce abnormal foot planar pressure during walking in neuropathy patients.

19. The device of claim 1, said device configured for use to prevent and/or reduce the formation of foot ulcerations in neuropathy patients.

20. The device of claim 1, wherein at least one of the one or more stimulators has adjustable stimulation strength.

21. The device of claim 20, wherein adjustment of the stimulator strength includes activating and/or deactivating a stimulator and is based on phase information of the gait of the patient.

22. The device of claim 20, wherein adjustment of the stimulator strength includes activating and/or deactivating a stimulator to provide a temporal stimulation pattern based on phase information of the gait of the patient.

23. The device of claim 20, wherein adjustment of the stimulator strength includes activating and/or deactivating a stimulator to provide stimulation in a pattern of frequencies based on phase information of the gait of the patient.

24. The device of claim 1, wherein the attachment mechanism includes one or more of suturing, stapling, one or more bone screws, or placement in a body cavity or bone.

25. The device of claim 1, wherein at least one stimulator is configured to stimulate a neuron of the patient.

26. The device of claim 1, wherein at least one stimulator is configured to stimulate an afferent nerve fiber of the patient.

27. The device of claim 1, wherein at least one stimulator is configured to stimulate a receptor of the patient.

28. The device of claim 1, wherein at least one stimulator provides electrical stimulation.

29. The device of claim 1, wherein at least one stimulator provides mechanical stimulation.

30. The device of claim 1, wherein at least one stimulator provides acoustic stimulation.

31. The device of claim 1, wherein the processor controls a stimulation frequency of a stimulator.

32. The device of claim 1, wherein the processor controls a stimulation duration of a stimulator.

33. The device of claim 1, wherein the processor controls a stimulation amplitude of a stimulator.

34. The device of claim 1, wherein at least one sensor is an acoustic sensor.

35. The device of claim 1, wherein at least one sensor is a tilt meter.

36. The device of claim 1, wherein at least one sensor is a goniometer.

37. The device of claim 1, wherein at least one sensor is a gyroscope.

38. The device of claim 13, wherein the rechargeable power source is configured to be recharged through an inductive coupling.

39. A method for treating a gait disorder of a patient, the method comprising the steps of:
a) generating a first set of electric signals indicative of gait-induced motion;
b) generating a representation of a gait cycle or portions thereof based on said first set of electric signals; and
c) using said gait cycle representation to provide feedback to the sensory system of the patient.

40. The method of claim 39, wherein said feedback is provided using a stimulator implanted in the body of the patient.

41. The method of claim 40, wherein said stimulator induces bone conduction.

42. The method of claim 41, further comprising operating said stimulator to induce bone healing and/or bone mineral density increase.

43. The method of claim 39, wherein the electric signals are generated based on lifting of a foot of the patient from ground during the human gait cycle.

44. The method of claim 39, wherein the electric signals are generated based on impact of a foot of the patient with ground during the human gait cycle.

45. The method of claim 39, wherein the electric signals are generated in response to acceleration in a direction that is transverse to a direction of the patient's gait.

46. The method of claim 39, wherein the feedback is by way of stimulators selectively activated based on prediction of phases of the gait of the patient.

47. The method of claim 39, wherein the electric signals are generated by a gyroscope.

48. The method of claim 39, wherein the electric signals are generated by an accelerometer.

49. The method of claim 39, wherein the electric signals are generated by a pieozoelectric sensor.

50. The method of claim 39, said method being used to treat gait and balance disorders in peripheral neuropathy patients.

51. The method of claim 39, said method being used to prevent and/or reduce falls in peripheral neuropathy patients.

52. The method of claim 39, said method being used to reduce abnormal foot planar pressure during walking in peripheral neuropathy patients.

53. The method of claim 39, said method being used to prevent and/or reduce the formation of foot ulcerations in peripheral neuropathy patients.

54. The method of claim 39, wherein said feedback is provided using a stimulator having adjustable stimulation strength.

55. The method of claim 54, further comprising adjusting stimulator strength by activating and/or deactivating the stimulator, said adjusting being based on phase information of the gait of the patient.

56. The method of claim 54, further comprising adjusting stimulator strength by activating and/or deactivating the stimulator to provide a temporal stimulation pattern based on phase information of the gait of the patient.

57. The method of claim 54, further comprising adjusting stimulator strength by activating and/or deactivating the stimulator to provide stimulation in a pattern of frequencies based on phase information of the gait of the patient.

58. The method of claim 39, wherein at least one of steps a), b) or c) is performed by a device that is attached to the body of the patient.

59. The method of claim 58, wherein the device is attached using a set of one or more screws and/or staples and/or suturing.

60. The method of claim 39, wherein said feedback is applied to a neuron of the patient.

61. The method of claim 39, wherein said feedback is applied to an afferent nerve fiber of the patient.

62. The method of claim 39, wherein said feedback is applied to a sensory receptor of the patient.

63. The method of claim 39, wherein said feedback is in the form of electrical stimulation.

64. The method of claim 39, wherein said feedback is in the form of mechanical stimulation.

65. The method of claim 39, wherein said feedback is in the form of acoustic stimulation.

66. The method of claim 39, wherein said electrical signals are generated by an acoustic sensor.

67. The method of claim 39, wherein said electrical signals are generated by a tilt meter.

68. The method of claim 39, wherein said electrical signals are generated by a goniometer.

69. The method of claim 39, wherein said electrical signals are generated by a gyroscope.
